(19) United States
(12) Patent Application Publication
(10) Pub. No.: US 2016/0321947 A1
(43) Pub. Date: Nov. 3, 2016

(54) SYSTEM AND METHOD FOR TREATING PATIENTS HAVING CONDITIONS THAT AFFECT WALKING

(71) Applicant: TWD SPORTS TECH, LLC, Annapolis, MD (US)

(72) Inventors: Brian R. Toronto, Annapolis, MD (US); Eduardo Salcedo, Indianapolis, IN (US); Aleksei Sebastiani, Indianapolis, IN (US); Joseph Malm, Mooresville, IN (US); James H. Wells, Oolitic, IN (US); Peter A. Altenburger, Carmel, IN (US)

(73) Assignee: TWD SPORTS TECH, LLC, Annapolis, MD (US)

(21) Appl. No.: 15/150,372

(22) Filed: May 9, 2016

Related U.S. Application Data
(63) Continuation-in-part of application No. 14/299,537, filed on Jun. 9, 2014.
(60) Provisional application No. 62/158,249, filed on May 7, 2015.

(51) Int. Cl. G09B 19/00; A61B 5/00; A61B 5/103

(52) U.S. Cl. CPC G09B 19/003; A61B 5/1038; A61B 5/0022; A61B 5/486; A61B 5/6807; A61B 5/7405; A61B 2562/0219; A61B 2562/0223

(57) ABSTRACT

A method for treating certain patients with walking impediments, such as stroke patents, are trained to walk more effectively by providing audio signals to them to reinforce them when they are walking properly, or to warn them to take remedial action when they are walking improperly. Sensors in an insert or shoe sense distribution of weight and foot strikes against a ground surface while walking, and an IMU senses foot movement. Outputs from these devices are analyzed by a processing system to detect when the patient is walking with an impediment, such as one that may cause stumbling and/or falling, and an audio warning is provided so the patient can train himself/herself to walk more properly. Such a device may also be used by a clinician to analyze foot problems in a rehabilitation setting, and devise a treatment protocol for the patient outside a rehabilitation setting.

### CROSS REFERENCE TO RELATED CONDITIONS

This application claims priority from Applicants pending patent application Ser. No. 14/299,537, filed Jun. 9, 2014. This application further claims priority from Applicants provisional patent application No. 62/158,249, filed May 7, 2015. Patent applications Ser. Nos. 14/299,537 and 62/158,249 are each incorporated by reference herein in their entirety.

### FIELD OF THE INVENTION

This invention relates generally to systems and methods for measuring parameters and positions of limbs of certain medical patients, and particularly to systems and methods for training patients who have medical conditions that adversely affect walking, balance and the like.

### BACKGROUND OF THE INVENTION

Positioning a body part in an appropriate orientation for various activities is an important thing to achieve in order to function normally. For example, in the simple act of walking, it is important that the feet and lower legs be appropriately moved and manipulated in order for a person to carry out daily tasks.

There are a number of medical conditions that impair or otherwise make it difficult to walk. For instance, after a stroke or other vascular accident of the brain, a patient will typically be weak on one side or the other, and may lose, to varying degrees, sensory perception that provides feedback with respect to proper foot placement while walking. In rare instances, a stroke or like may be bilateral and affect both sides of a patient's body with corresponding loss of sensory feedback. In these situations, a patient may need to relearn how to walk. Particularly with respect to stroke patients, who as noted are typically weak on one side or the other, walking flat-footed, or with “dropfoot” is a common affliction that can cause stumbling and attendant falls. To avoid these accidents, many stroke patients walk while looking at their feet to determine their foot placement, which in turn can cause other accidents. In addition, stroke patients tend to put more weight on their “good” leg and foot, as opposed to balancing while standing still with a 50/50 body weight distribution for their feet. As should be apparent, this increases where and tear of bones and tendons in their good feet and legs. In other situations, disease processes, such as diabetes, may cause nerve damage or neuropathy due to impaired circulation with corresponding loss of the ability to properly sense where their feet are and how they are oriented while walking. In yet other situations, accidents may cause damage to bones, joints and nerves, which damage interfering with proper placement of the feet. Related to this is joint replacements where hips, knees and the like are replaced, which may result in abnormal placing of the feet while walking. In addition, leg amputees such as wounded veterans must relearn how to walk when fitted with prosthetic legs and feet. Further yet, children with cerebral palsy may need to learn to walk, or may need to relearn how to walk after developing improper walking habits.

In determining proper foot placement of such patients during walking in a rehabilitation setting, several parameters should be measured. One such parameter is foot orientation. The foot is essentially movable about three axes that can be analogized, by way of example, the roll, pitch and yaw of an aircraft. Here, roll would be movement of the foot where opposed sides of the foot are moved up and down in opposition to each other, pitch is illustrated as the toes and distal portion of the foot moving in up and down directions, while yaw is movement of the foot around an axis of the lower leg bones.

When a person who walks normally takes a step, their feet are placed in appropriate roll, pitch and yaw orientations in order to both support and balance the body and propel it in a desired direction. The heel is placed first with the foot in a toe up orientation, and as the stride progresses the middle outer portion of the foot is loaded, and the ball of the foot and toes roll forward over the ground or other surface to be traversed. As the heel of the other foot engages the ground surface, the heel of the first foot lifts off the ground and weight shifts to the ball and toes of the first foot as the first foot “pitches” upward as a result of being behind the weight of the person. When walking straight ahead, the long axes of both feet are maintained parallel or aligned with the direction of walking.

The following definitions define triplaner movements of the foot, which are transverse motions (abduction/adduction), frontal motions (inversion/eversion) and sagittal motions (dorsiflexion/plantarflexion):
Dorsiflexion and plantarflexion relate to “pitch” of the foot. Dorsiflexion is movement of the toes in an upward direction, and plantarflexion is movement of the toes in a downward direction.

Abduction and adduction refer to rotation of the foot about an axis defined by the leg bones, and would correspond to yaw in an aircraft. Abduction is movement of the front portion of the foot medially, or inward, while adduction refers to movement of the front portion of the foot laterally, or outward.

Eversion and inversion are analogous to roll of an aircraft wherein eversion is movement of the plantar surface (bottom of the foot) laterally, or in an outward direction, while inversion is movement of the plantar surface medially, or in an inward direction. The above movements of the foot can combine into many different triplanar positions while walking. For example, and with respect to abnormal positions, pronation is a condition wherein the ankle protrudes or is rolled outwardly, and can lead to a bowlegged stance. It is actually a combination of eversion, dorsiflexion and adduction. Supination is the opposite wherein the ankle protrudes or is rolled inwardly, and can create a “knock knee” stance. Supination is a combination of inversion, plantar flexion and abduction.

In a rehabilitation setting, a clinician working to teach a patient how to walk or learn how to walk again must teach the patient how to place and orient their feet while going through the motions that involve walking. Currently, such teaching may be facilitated by data obtained by pressure sensors that the patient walks on, the pressure sensors coupled to computers. The sensors are incorporated into pressure pads that, in some instances, are of a size so as to allow a person to stand on the pad to allow analysis of how the patient is balancing himself/herself and determine weight distribution on the feet. In other instances, the pressure pads are installed on a walkway or even on a treadmill-type device so that the patent may walk on them in order to gather information about how the feet are oriented and train the patient as the patient walks. As noted, pressure pads are also coupled to a computer via a user interface that typically involves a dedicated monitor, keyboard and mouse, and perhaps other specialized electronic equipment.

While pressure pads serve their purpose, their use is limited to rehabilitation and similar settings where they are used, and clearly cannot indicate what is happening with a patient's feet as they go about their daily activities outside the therapeutic setting. Where a patient is assigned rehabilitation tasks to undertake, compliance cannot be monitored outside the rehabilitation setting, which leads to higher medical costs. In addition, pressure pads can be expensive, costing from thousands of dollars to tens of thousands of dollars or more at today's prices.

Shoes and shoe inserts having pressure sensors are known. Perhaps exemplary of such shoes and shoe inserts is U.S. Pat. No. 9,089,182, issued Jul. 28, 2015, to Schrock et. al., and which is incorporated herein in its entirety by reference. The Schrock patent discloses throughout shoes and shoe inserts for athletes and various embodiments thereof, the shoes and shoe inserts having pressure sensors of various materials incorporated therein. An electronic module built into the shoe or shoe insert collects data from the pressure sensors, and a communications port transmits the data to a device for performing analysis of the data. In a simplest embodiment, data is collected by the module and transmitted to another device for processing. In another embodiment, at least some processing is done by the processing system in the module. In one embodiment, active, real-time feedback is provided to a wearer by way of a vibration element, although it is unclear from Schrock exactly what triggers the feedback, how the feedback is used and what the feedback would do for an athlete. Also, as noted, since the shoes and inserts, and sensor orientation of Schrock are intended for athletes and configured toward athletic performance parameters, it is unclear from Schrock how such shoes and inserts could be used to help someone relearn to walk after having a stroke or how they would be used to help the person with abnormal medical conditions or disease processes related to walking. Further, since the vibration element of Schrock would most probably be a small motor that rotates an eccentric weight, such as found in cell phones, a battery powering the electronic module of Schrock would be quickly drained as a patient walks around during their daily activities.

From the foregoing, it is apparent that a shoe insert or shoe is needed that provides instantaneous audio feedback to a patient, the feedback indicating normal or abnormal foot orientation, foot placement and foot movement. Degree or extent of improper foot placement, foot orientation and foot movement may also be indicated. Such audio feedback would allow many patients to look ahead while walking instead of looking at their feet to ascertain position of their feet, which as noted is the case with many stroke patients. As such, an insert or shoe of the instant invention may prevent those patients having problems with their gait or positioning their feet from falling and sustaining further injuries, and would improve their quality of life, in addition to assisting clinicians in rehabilitation settings to train affected patients how to walk.

### BRIEF DESCRIPTION OF THE DRAWINGS

FIG. 1 is a schematic view of an insert of the instant invention showing placement of pressure sensorsin or on the insert.

FIG. 2 is an illustration of an insert of the invention showing mounting of an electronics module behind a heel of a patient.

FIG. 3 shows how the insert of FIG. 2 fits over a heel of a shoe.

FIG. 4 illustrates placement of an electronics module of the invention on a tongue of a shoe.

FIG. 5 shows integration of an electronics module of the invention into or integral with a shoe.

FIG. 6 shows a block diagram of one possible embodiment of an electronics module of the invention.

### DETAILED DESCRIPTION OF THE DRAWINGS.

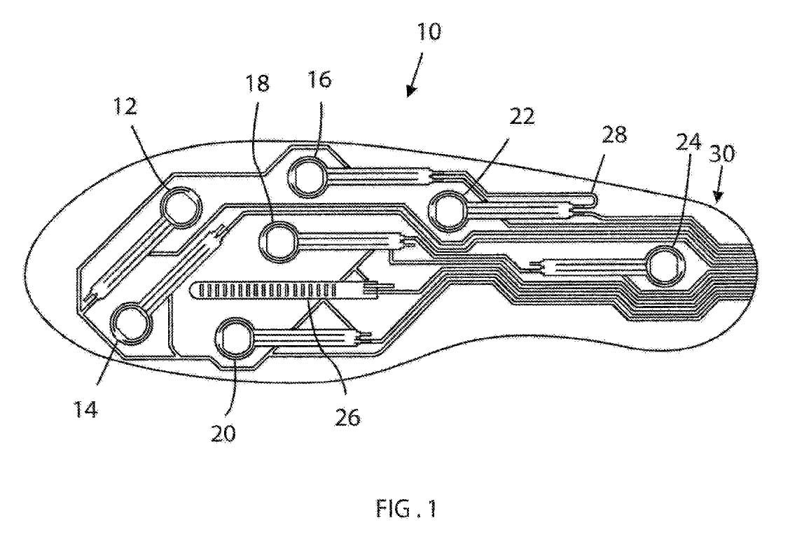

Referring initially to FIG. 1, a shoe sole portion 10 of a shoe insert of the invention is illustrated. Sole portion 10 is generally shaped similar to an insole of a shoe and is positioned in a shoe where an insole would be positioned. However, in some embodiments, portion 10 is configured to be flat, as opposed to an orthotic insole that typically would be curved in order to correct certain conditions of the foot. Such a flat configuration does not distort pressure sensors in the insert, or alter pressure readings therefrom. A plurality of pressure sensors are mounted to or otherwise associated with insert 10.

In accordance with the invention, sensors 12, 14 are located under toes of a patient, sensors 16, 18 and 20 are located across and under a ball of the foot of the patient, sensor 22 is located as shown under the outside of the foot adjacent the arch of the foot, and sensor 24 is located under a heel of the patient. The number and location of sensors 12-24 is optimal for detecting both static and dynamic weight loading of associated portions of a foot, although positions of the sensors may be varied somewhat, and in some instances only two sensors may be needed under a ball of the foot, one to the inside and one to the outside of the ball of the foot.

Portion 10, also designated as insert 10, may be constructed of any flexible, pliable and durable material, which may be a plastic, foam or organic material that will hold up under the stresses of walking. In some embodiments, insert 10 may be constructed of layers of material in order to accommodate sensors 12-24. Materials used to fabricate the individual layers may include natural or synthetic leather, natural or synthetic textiles, polymer sheets, polymer films, mesh textiles, felts, non-woven polymers, rubber materials and plastic materials. The layers may be stitched, or bonded together by adhesive, or ultrasonically or heat welded together. In some embodiments, polyurethane, ethylvinyllactate or other materials, such as phylon, phylate, that compress to attenuate ground-striking forces may be used. In other embodiments, portions of or all of the insert may be constructed from polymer foam materials or other flexible plastic materials may encapsulate or include various other elements, such as one or more fluid or gel-filled bladders that enhance comfort where patients are suffering from other foot conditions, such as bunions and bunionetts. In some exemplary embodiments, the insert 10 includes a flexible printed circuit board material, such as Pyralux™, available from DuPont™. Pyralux is a copper clad flexible circuit board material that can be etched as shown to form circuit runs, for example 28, that are soldered to sensors 12-24 and a coupled to an electronics module that may be a power and control module (not shown). In FIG. 1, circuit runs 28 from all the sensors are shown terminating at heel 30 where such a power control module would be attached, either directly or via other conductors such as ribbon cables. Here, the insert may be formed from a bottom layer of one or more of the aforementioned materials, with the Pyralux mounted to an upper surface thereof. In any case, insert 10 is constructed to incorporate sensors 12-24 such that the sensors accurately detect weight from various portions of the foot placed upon them during walking of the patient.

Sensors 12-24 may themselves are configured to sense force or weight applied by feet of the patient, and may be constructed and incorporated into an insert as disclosed at the paragraph bridging cols. 9-10 of the incorporated Schrock patent (U.S. Pat. No. 9,089,182, col. 9 lines 43-67, col. 10 lines 1-26, sensors 16 in the drawings), and at the paragraph bridging cols. 10 and 11 of the Schrock patent (col. 10 lines 27-67 and col. 11 lines 1-6). In addition, suitable force sensors may currently be purchased from many sources, such as TEKSCAN™, which sells force sensors that can be made to any size, are flexible and are thin, on the order of 0.008 inches. Also, any of the aforementioned force sensors may be integrated with etched Pyralux™ circuit board material to form an insert with the sensors, with circuit runs etched as shown in FIG. 1, or an insert may be constructed along the lines of the teachings of the Schrock patent and having circuit runs etched or incorporated in a layer of the insert, or in a single layer insert. In any case, sensors 12-24 sense varying amounts of weight applied to them, with outputs provided to an electronic processing system either in or mounted to the insert or exterior of the insert.

Location of sensors 12-24 is such that each sensor provides an indication of weight from the portion of the foot that bears on a respective sensor. Taken together, and with reference or comparison to static forces applied by a normal foot, abnormal conditions of the foot may be ascertained while a patient is standing in a fixed position. In addition, and compared to dynamic forces applied to the sensors during walking, data is obtained that can diagnose and provide real time audio feedback to a patient while the patient is standing still or walking in order for the patient to either learn to balance properly or walk correctly or assist the patient with proper foot placement while walking. As such, sensor 12 is located generally under the two outermost, smaller toes, with sensor 14 generally under the big toe. Sensors 12, 14, when the patient is standing still, may indicate balance problems, such as whether the patient is “weaving” or rocking back and forth in any direction by using his/her toes more than normal to maintain a balanced orientation. When walking, sensors 12, 14 indicate when the toes strike the ground and when the toes lift off the ground. Any abnormal weight distribution between sensors 12, 14 can be an indication of eversion or inversion.

Sensors 16, 18 and 20 are located generally from side-to-side and slightly angled as shown across the ball of the foot, with a flex sensor 26 extending lengthwise with respect to the foot and located generally as shown between sensors 18, 20. Flex sensor 26 is basically a ribbon-shaped sensor that senses flexure of the foot while walking. Sensor 26 may indicate whether the patient is walking “flat-footed”, i.e. planting the foot generally flat on the ground rather in a heel first manner. Degree of such flat-foot walking can be indicated by degree of flexure of 26.

Sensors 16, 18 and 20 also can indicate dorsiflexion and plantarflexion, as well as timing as to whether the parts of the foot are planted in proper order, i.e. heel first, then the middle of the foot followed by the ball of the foot, and lastly the toes. Sensor 22, located across the foot opposite the arch of the foot, is involved with indicating timing of the foot parts as they strike the ground and progressive shifting of weight between the heel, ball of the foot to the toes. Likewise, sensor 24 indicates a heel strike, which should be the first part of the foot to strike the ground when walking, and can indicate to a stroke patient that they either need to lift their legs more or alter the pitch of the foot so that the toes are more elevated.

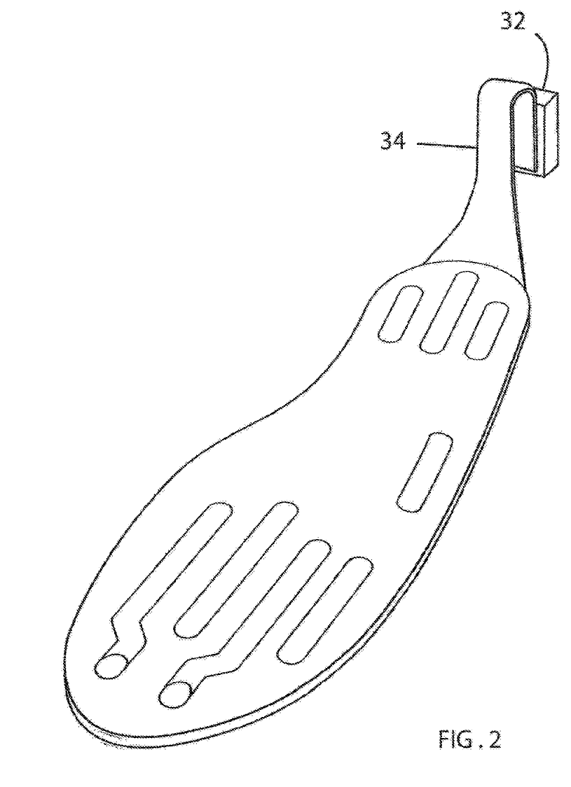

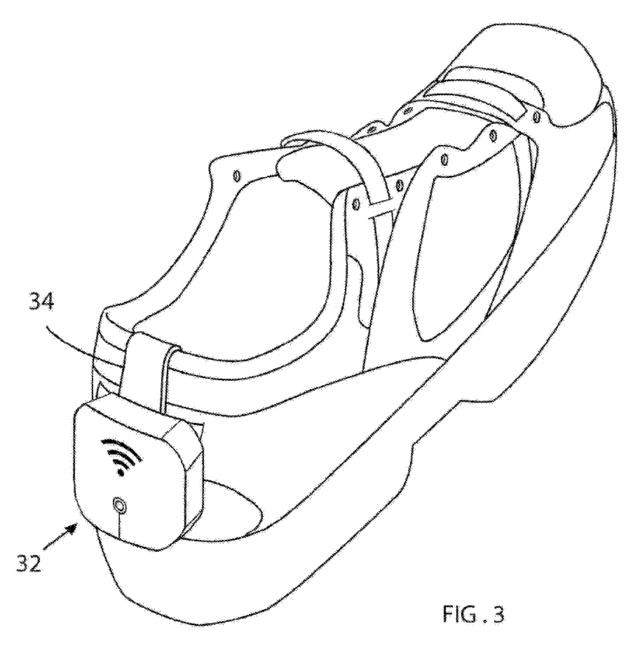

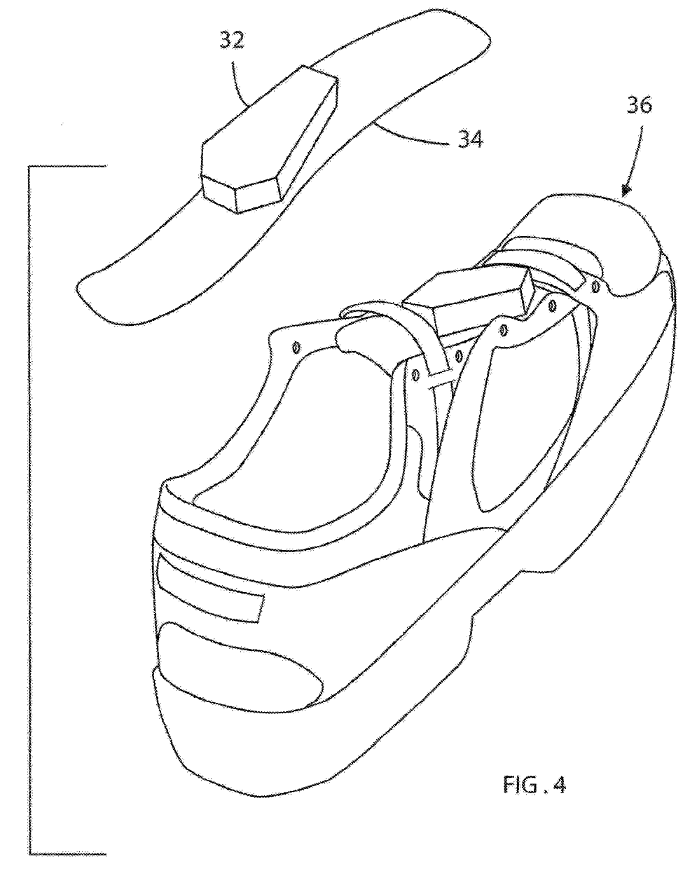

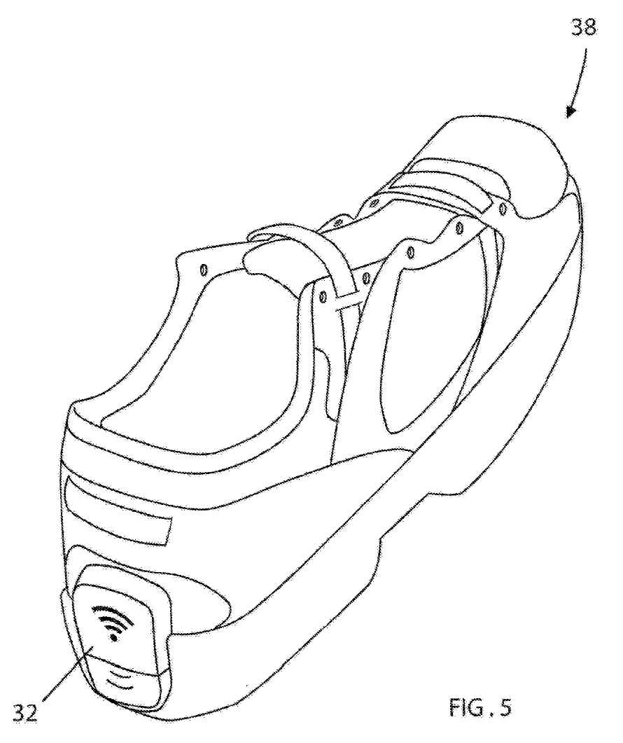

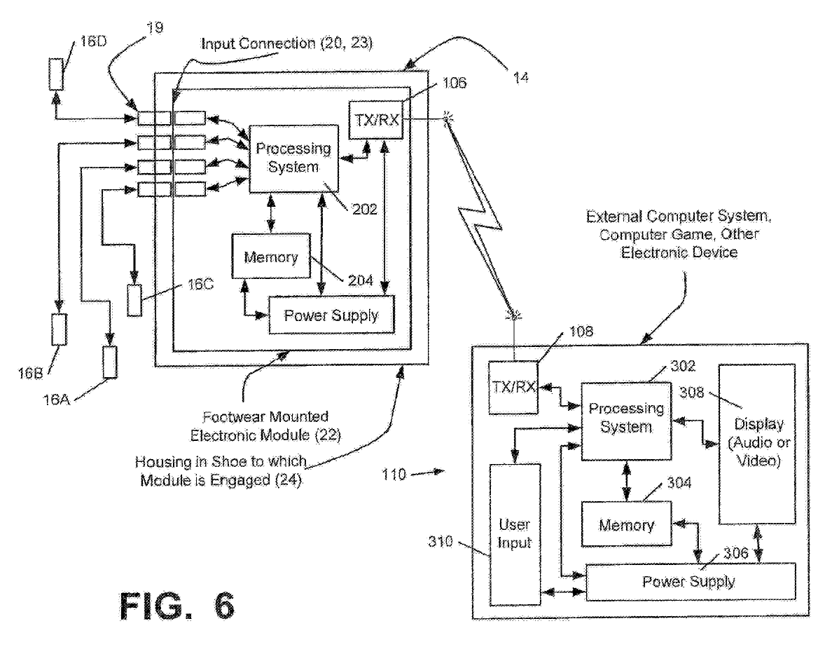

In some embodiments, sensors 12-24 are connected to a communications port that transmits data to an electronic module mounted to insert 10, or in or to a shoe, with the insert against an interior lower surface of the shoe so that the various parts of the foot are above the appropriate sensors as described herein. For instance, the port may communicate with an exterior module as disclosed for port 14 in the Schrock patent at Col. 11 lines 7-52, lines 62-67, and be configured as disclosed in the Schrock patent at FIG. 6, Col. 14 lines 62-67 relating to port 14, interface 20, electronic module 22, sensor leads 18 interface 19, data transmission/reception system 106, electronics module 22, footwear structure 100 and other associated components. An exemplary circuit for a communications port and/or electronic module is shown in FIG. 6 of Schrock as further including a processing system 202, memory 204, a power supply, which may simply be a battery, and a transmitter/receiver 106 that allows any form of communication with an exterior device, such as an ear device worn by a user of the insert. The circuit of FIG. 6 is described in more detail at Col. 14 lines 62-67 and the entirety of Col. 15 of Schrock. Applicant's electronic module may be directly connected to an insert 10 and fixed to insert 10 (diagrammatically shown in FIG. 2) by a looped portion that fits over a rear of a shoe as shown in FIG. 3. In other embodiments, such as shown in FIG. 4, an electronic module or power and control module 3 may be mounted to a tongue 34 of a shoe 36 (shown separated), and communicate with sensors 12-24 and powered by a small battery-powered micro-transmitter in the shoe 36 or insert 10 to a receiver in module 32. Data from module 32 is then transmitted to a user device, such as an earpiece or other electronic device. In another embodiment shown in FIG. 5, a power and control electronic module 32 may be mounted directly to a shoe 38, and be hard-wired to sensors 12-24 mounted in the shoe sole itself. In other embodiments, an electronics module 32 may be mounted as disclosed in the Schrock patent at Col. 9 lines 6-42 with respect to FIGS. 4-5 showing midsole 131, foot contacting member 133, cavity or recess 135, port 14, outsole 132, shoe 100, sole 130, sensors 16 and leads 18. The electronics module at the foot may be powered by one or more batteries, either rechargeable or non-rechargable. Where a rechargable battery is used, a charger may be plugged into the module, or an inductive loop in the insert or module may be used so that the module or insert is simply placed on, over or near a charging loop in order to charge one or more batteries in the electronic module.

In another embodiment, and as disclosed in the Schrock patent at Col. 16 lines 20-44, relating to FIG. 6 showing electronic module 22, footwear 100 and sensors 16, activation of the system or parts of the system may be accomplished by tapping a heel or toe in a predetermined sequence. Applicant contemplates using such heel taps, toe taps or both, in addition to activating the system or parts of the system, but also as a means of communication. Here, in a military or similar application, a soldier or other person may use such taps of sensors located underneath a toe, heel or both to tap a sequence to signal his/her location, status, indication of an attack or another pattern to notify military or other operations services. In a simplest embodiment, such signaling could be accomplished by a simple code, such as Morse code or other code, transmitted by a short-range or wired transmitter in or coupled to the insert, and in some embodiments, transmits the code to a radio or other communication device carried/worn by the soldier. In a patient setting, such a feature of tapping out a code may signal an emergency situation to emergency responders, such as where a patient falls and can't get up. The code can be transmitted by an integrated transmitter in a power and control module or electronics module of the invention as previously disclosed in the incorporated Schrock patent, or by a more powerful transmitter worn by the patient and which receives signals from the insert or shoe. A receiver of such tapping signals may be a cell phone, tablet computer desktop computer, a device coupled to a wired telephone line or to another device coupled to emergency services. In some embodiments, toe or heel tapping may not be available, as where a patient falls and injures himself/herself, so it is contemplated that a patient, soldier or other person may use toe presses against a sensor, such as a big toe pressing against sensor 14 (FIG. 1) to transmit a code or summon emergency services. In any of these tapping embodiments, GPS, GLONASS or other location service would be integrated into either the processing system at or coupled to the insert or shoe, or use a GPS system or the like in a cell phone, tablet computer or other system worn or associated with the person so that emergency service people would know the location of the person tapping or toeing out a signal.

While useful information is gained by how the parts of the foot strike the ground and when they lift off the ground, information relating to extent of abnormal motions is gained if it is known how the foot is oriented and direction it is moving between steps. Accordingly, it is contemplated that Applicant's shoe insert include an inertial measurement unit (IMU) that detects orientation and direction of movement of the feet. One such IMU device may be a 9-axis IMU that includes a 3 axis gyroscope, a 3 axis accelerometer and a 3 axis magnetometer, part number LSM9DS1, available from SparkFun (Amazon.com). Such a device, when incorporated into an insert of the invention, provides data to a processor, such as the processing system 202 of Schrock, the data related to orientation, direction and accelerations of a foot during walking. This is important when dealing with abnormal parameters related to extent of motions of the foot that involve roll, pitch and yaw of the foot. While a 9-axis IMU is disclosed, an IMU that incorporates only a 3-axis gyroscope and 3-axis manometer may be used. The IMU package is mounted so that the inertial reference frame, or axes of the gyroscopes, are aligned to sense triaxial movement, or roll, pitch and yaw, of the foot. This is very similar to a 3-axis gyroscope, or 6 or 9 axis IMU of a hobby or toy drone helicopter. Applied to an inert of the instant invention, a first gyroscopic axis of an IMU is aligned with the long axis of the foot and indicates abduction/adduction (yaw) orientation of the foot, and senses the extent or degree to which toes of the foot are not pointed along a reference direction. Similarly, when eversion/inversion (roll) is present, a second gyroscopic axis aligned across the foot senses when the foot is rolled as it is lifted off the ground by comparing its position to a second reference position. Again, this gyroscopic axis senses degree or extent of rolling motions of the foot. The third gyroscopic axis is oriented generally parallel to the leg bones when the patient is standing straight, and senses extent of dorsiflexion and plantarflexion (pitch) as compared to the standing reference position. The gyroscopic axes are typically calibrated with the feet planted flat on the ground, or may be calibrated simply by putting the Insert or shoe flat on the ground, with this position stored as a baseline position of the feet for comparison to orientations of the foot while moving. Where there are two inserts, one for each foot, the three axis gyroscopes therein may be calibrated by placing the inserts in a known orientation for normal feet and storing that position as a basis for comparison for when the user is walking. In the instant invention, such tilting away from “normal”, or calibrated reference positions produces sound or changes of sound provided to a patient, as will be further described

The three axis magnetometer provides outputs that allow directions the foot is travelling to be ascertained. As such, the 3-axis gyroscope provides indications as to tilting motions of the foot, while the 3-axis magnetometer provides indications of directions the foot is traveling. These data outputs from the magnetometer are combined with the gyroscopic outputs to determine how a patient's foot or feet are moving. Here, the magnetometer senses when the foot is travelling forward and upward, and when some patients swing their legs outward or inward as they walk. A microcontroller coupled to the 3 axis gyroscope and 3 axis magnetometer can detect any of the triplanar movements of the foot as the foot is moved forward, lifted upward and lowered.

Accelerometers in the IMU can provide parameters such as a number of steps a patient may take during a day away from a rehabilitation setting. This allows a clinician to determine compliance with any given rehabilitation program and how a patient is progressing. Such accelerometers also provide frequency of movement and vigorousness of movement. In addition, accelerometers can serve as predictors of wear and tear on joints by determining whether a patient is planting his/her feet too hard or otherwise moving too abruptly.

With respect to FIG. 6 of Schrock, Applicant's processing system and/or port system would be similar in that port of other electronic module 14 is coupled to a shoe or shoe sole insert as described herein, and would include at least processing system 202 having an appropriate number of connections 20, 23 to accommodate sensors 12-24 (Applicant's FIG. 1). Processing system 202 is configured to sense static and dynamic weight loading applied to sensors 12-24, most probably in an analog manner. An A/D converter associated with or integrated in processing system 202 converts the analog signals from sensors 12-24 to digital signals with sufficient resolution so that weight changes during walking are accurately resolved. An associated memory 204 (FIG. 6 of Schrock) includes permanent or non-volatile storage for program boot data, and nonvolatile but erasable program data for storage of patient parameters, which would include at least weight, and possibly other parameters such as height, and data used by the program that is customized for that particular patient. This is particularly useful where an insert and associated module is to be used for more than one patient. Here, after one patient is fully treated, their information may be erased and and the next patient's information and calibration data is programmed into the electronics module. Customizable data for a particular patient could include calibration information for the IMU sensors, and parameters unique to that particular patient as determined by a clinician in a rehabilitation or similar facility. Here, by way of example, where a stroke patient has dropfoot in one foot, i.e. walking flatfooted on that foot, and also favoring a weak leg and foot, then processing system 202 and memory 204 would be programmed to detect when the patient is walking flatfooted by a combination of sensors 12-24 recording foot strikes that do not progress in the normal heel-to-toe manner, but rather all portions of the bottom of the foot strikes the ground almost simultaneously. In combination with inputs from sensors 12-24, the IMU would report that the foot is oriented and moved in a relatively flat orientation rather than a toe-up orientation. This combination of sensor inputs would cause processing system 202 to generate an output that is transmitted to a patient earpiece, symbolized by electronic device 110 of Schrock, to provide feedback in the form of a sound to the patient indicating that he/she is walking with dropfoot. Sensors 12-24 would indicate when an unbalanced weight distribution is applied to inserts under both feet, with the electronic module reacting to also provide sound indicating the unbalanced condition. Such a sound could be any sound the patient would respond to, and could be configured as negative feedback such that the sound is provided when dropfoot is present, or as positive feedback so that the sound is provided when the patient is correcting and dropfoot is not present. The sound may be varied to indicate degree or severity of the dropfoot, such as varying frequency of repetitive sounds such as clicks, chirps, intermittent buzzing and so forth, or the intensity of the sound may be increased or decreased, such as where music, musical tones or the like are provided as feedback. Where there are multiple problems as described, different tones or sounds are provided, each indicating a different respective condition to be corrected. Thus, dropfoot may be treated by one sound, frequency of sound, intensity of sound or the like, and unbalanced weight distribution may be treated similarly by a different sound. In some embodiments, the sound may be a voice that may tell or warn the patient that he/she is walking in an abnormal manner, or how to correct abnormal walking. As such, the warning generated for dropfoot may say “lift the toes of your right foot”, or “Lift your right leg higher”. Where a patient is favoring one side of the other, the voice may say “shift more weight to your left foot” and “OK, you are balanced properly”. As should be apparent, any audio warning or notification can be provided.

In other embodiments, rather than having customized patient regimen as described, common conditions related to stroke patients may be input to an EPROM or EEPROM integrated into programming system 202 in a “one size fits all” manner. As such, patient information would be provided during calibration and initial setup, and the programming system 202 would provide feedback in accordance with any abnormal walking activity in its respective nonvolatile memory. Here, where a patient has a deformity or condition that prevents normal orientation and/or movement of one or both feet, such as where eversion or inversion are permanently present, the reference frame of the IMU can be adjusted to account for the unusual orientation of the feet and only provide feedback in accordance with a treatment plan for treating a different walking condition. For instance, where a patient has permanant inversion of about 4 degrees of so, the gyroscope axis that detects eversion/inversion could be offset using software so that the 4 degrees of inversion would be indicated as 0 degrees of inversion. In a hobby or toy drone helicopter analogy, this would be similar to or the same as adjusting trim of the helicopter to compensate for drift of the helicopter from one side to another when no control inputs are present. As such, and since people's feet are likely slightly different anyway, minor corrections to the reference frames of the IMU may be made to compensate for such differences.

With respect to a patient earpiece, and in a simplest embodiment, a simple ear bud speaker may be wired using flexible wire from the electronic module 14 (FIG. 6) of Schrock or Applicant's electronic module 32 (FIGS. 2, 4), which is located at the shoe of the patient, to the ear bud speaker. In this instance, processing circuitry, including sound generators and amplifiers for the speaker or speakers would be located in electronic module 14. In other embodiments, an intermediate electronic module wired to a shoe or insert may be worn elsewhere, such as on a waist belt, ankle strap or the like, with signals from the insert or shoe by wire or wirelessly from the intermediate module to speakers at the patient's ears. Such embodiments would allow for larger batteries in the intermediate module, and correspondingly longer battery life, and in some instances more processing power by allowing more space for processing circuitry, as where other biometric parameters, such as temperature, blood pressure, heart rate heart rhythms and other parameters are being monitored. In yet other embodiments, a low power Bluetooth transmitter in module 14 may be used to transmit audio signals to an earpiece receiver, or a low power wireless body area network (WBAN) transmitter/receiver pair may be employed to transmit audio from the shoe to a receiver at the ear. In other embodiments, signals from the shoe or insert may be sent to a personal electronic device, such as a cell phone, tablet computer or the like, which in turn provides audio feedback signals, as by an ear speaker or Bluetooth-type device, to the patient. In addition, since any common radio transmission protocol and associated transmitter in electronic module 14 may be used, the electronic module 14 at the patient's shoe can communicate directly to any device configured with a receiver, such as a tablet computer, desktop computer, cell phone or the like. This would allow use of the instant invention in a rehabilitation facility without any special provisions other than the patient walking into the facility wearing the insert or shoe, with a clinician simply using his/her computer (fitted with an appropriate radio receiver) to receive signals from module 14 worn by the patient. In this instance, and where a module 14 is also fitted with the appropriate radio receiver, control signals may be sent from the clinician's computer to electronic module 14 worn by the patent in order to adjust responsiveness of module 14 in accordance with improvement made by the patient in his/her walking ability. Further, a storage medium, such as a micro SD card or the like, may be used to store data in module 14 for later retrieval and use by a clinician, or the clinician may retrieve data stored in module 14 via the appropriate receiver coupled to his/her computer. Further yet, where data from the shoe or insert is transmitted to a cell phone or tablet computer, processing of the data may be done using an applet in the cell phone or computer, which in turn provides audio feedback signals to the patient. In this case, the only data needed from a transmitter located at the shoe or insert is the data from sensors 12-24 and the outputs from the IMU. The applet in a cell phone or the like is loaded into the cell phone and calibrated for a given patient by a clinician at a rehabilitation facility so that the cell phone, using the applet, does all the processing and providing of audio feedback. Such an applet could easily be written for open source Android cell phones, which would allow for cross platform use for most cell phones and use Bluetooth to communicate with a Bluetooth or WBAN transmitter where the foot sensors are located, and provide audio feedback to the patient via an earbud or headset device.

In some embodiments, a module 14 is provided for each foot of a patient, and communicate, as by WBAN radio signals, with processing systems in respective inserts, i.e. the inserts are talking to each other, or to an applet in a cell phone, a program in a tablet computer, wearable computer, computer at a rehabilitation center or the like. In this instance, information related to how both feet move in different situations and in real time becomes available to the clinician, and audio feedback related to both feet may be adjusted to accommodate such different situations. For instance, one of a patient's feet may be moving or be oriented abnormally when a patient is walking faster than when walking slower. In another situation, a patient may be correcting a limp, with audio feedback provided accordingly. Another example is where a person is favoring one leg over the other. Here, the length of time one of the patient's feet is on the floor can be compared to the other foot, and audio feedback adjusted according to a treatment protocol.

Another situation may arise where it is useful to have information from both feet is where amputees such as veterans are learning to walk or even run with one or even two prosthetic legs and feet. Here, since there is no sensation from the prosthetic foot, the amputee must learn how to position and orient the remainder of his/her leg in order to properly orient and move the prosthetic leg and foot with respect to the other foot. In this instance, an insert having sensors 12-24 and worn in a shoe of a prosthetic foot would be invaluable in teaching such an amputee how to use the prosthetic limb while reducing length of time necessary to train such an amputee. In addition, since there is no requirement to shape a prosthetic limb and foot like a normal foot, a prosthetic limb and foot may be configured for a specific purpose, and interchangable with other prosthetic limbs for other purposes. For instance, a curved blade-like prosthetic may be used for running activities, and would have pressure sensors and an IMU configured for use for training with that type prosthetic. A more normal-looking leg and foot prosthetic with an insert and module 14 as shown in FIG. 1 may be worn during daily activities. Each of these situations would require the leg/legs having the prosthetic device to be moved differently, with the amputee being trained accordingly. Having thus described the invention and the manner of its use, it should be apparent to those skilled in the relevant arts that incidental changes may be made that fairly fall within the scope of the following appended claims, wherein we claim:

1. A medical device for determining position and orientation of a body part, comprising:
a sensor portion and a processor portion, the sensor portion including a plurality of sensors that are interactive with the body part to determine the amount of pressure that is placed on this sensors;
an accelerometer to measure the speed of the body part;
a gyroscope to measure the yaw, pitch and roll orientation of the body part; and,
a magnetometer to measure the geographic directional orientation of the device; and
wherein the processor receives the data from the sensors and includes a communications component for communicating the processed data to a remote processor for further processing.

2. A method for treating a patient with an abnormal gait comprising:
sensing weight applied to portions of at least one affected foot of said patient against a ground surface while said patient is walking,
sensing direction of motion of said at least one affected foot while said patient is walking,
sensing orientation of said at least one affected foot while said patient is walking,
analyzing at least said weight, said direction of motion and said orientation of said at least one affected foot,
developing an audio feedback signal to said patient, said audio feedback signal configured to train said patient to reduce or eliminate said abnormal gait.
